(19) United States
(12) Patent Application Publication
(10) Pub. No.: US 2022/0162585 A1
(43) Pub. Date: May 26, 2022

(54) SYSTEM AND METHOD OF NUCLEIC ACID EXTRACTION

(71) Applicant: Frank Leo Spangler, St. George, UT (US)

(72) Inventor: Frank Leo Spangler, St. George, UT (US)

(21) Appl. No.: 17/456,215

(22) Filed: Nov. 23, 2021

Related U.S. Application Data
(60) Provisional application No. 63/117,270, filed on Nov. 23, 2020.

(51) Int. Cl. C12N 15/10; C12Q 1/6806; B01L 3/00; B01L 7/00

(52) U.S. Cl. CPC C12N 15/1013; C12Q 1/6806; B01L 2200/0668; B01L 7/00; B01L 2300/18; B01L 3/5021

(57) ABSTRACT

A method of nucleic acid extraction utilizes small magnetizable beads (e.g., iron, cobalt, nickel, etc.) that react to the presence of a magnetic field by participating in a paramagnetic flux. The magnetizable beads clump together in a mass inside of a reaction vessel. The reaction vessel can be different shapes and sizes, but it is typically a cylindrical test tube. A magnet is positioned proximal to the reaction vessel with the beads inside. By rotating the magnet around the tube (or spinning the tube), the beads will follow the magnet around the tube due to the magnetic flux, creating a grinding or macerating effect while mixing the contents. When biological samples are also placed into the reaction vessel with the magnetizable beads, they become homogenized in a solution in the reaction vessel as the magnet moves around the vessel.

### CROSS-REFERENCE TO RELATED APPLICATIONS

This application claims the benefit of U.S. Provisional Application Ser. No. 63/117,270, filed on Nov. 23, 2020, which is incorporated herein by reference.

### TECHNICAL FIELD

The present disclosure relates to biotechnology. More particularly, the present disclosure relates to a system and method of nucleic acid extraction.

### BACKGROUND

Polymerase Chain Reaction (PCR) is the gold-standard used in molecular diagnostics. However, there are still a few bottlenecks that slow down the PCR process. One such bottleneck is sample preparation.

For PCR to work, there must be purified nucleic acids in the reaction that match the primers used for detection. The process of getting the DNA or RNA out of a specimen that has a mixture of biological molecules is called nucleic acid extraction or, simply, sample preparation. There are several different extraction processes and chemistries, but all of them produce the same result—purified nucleic acids ready for PCR.

Currently in the art, it usually takes 15-60 minutes to process a specimen to extract the purified DNA or RNA. Depending on specimen type, that time could be significantly longer. Viruses and gram negative or cell wall deficient bacteria can be fairly easy to extract RNA and/or DNA from the specimen. Regarding gram positive bacteria and plant cells, the extraction process becomes more difficult due to the thicker and/or tougher cell walls found in these specimens. Fungal specimens are even more difficult due to their protective cell walls. Large tissue specimens pose another difficulty. While the individual cells found within a tissue sample may be disrupted quite easily, only the cells on the surface of the tissue sample are readily available to the extraction reagents. Cells found deep within the tissue sample are protected from the extraction reagent, resulting in lower yields of RNA and/or DNA unless something is done to pre-process the tissue specimen. One example used to pre-process the tissue is an enzymatic digestion with proteinase K. This process can take 12-24 hours to complete, depending on sample size of the tissue specimen.

Spores pose an even greater difficulty for sample extraction and purification. Bacillus anthracis, the causative agent in Anthrax, is an example of a bacterium that produces spores. These spores are essentially armor coated hibernation capsules that are extremely hard to disrupt to release any RNA and/or DNA.

As a result of the varying difficulty of specimens, there are numerous methods in the art for extracting nucleic acids. A user must therefore learn various methods and obtain several devices and solutions to process different specimens. This is time consuming and expensive.

Therefore, there is a need for a quick and simple extraction method that can handle a range of specimens, including easy to difficult, without deviation from the method, and it would produce highly purified nucleic acids in just a matter of minutes. The present disclosure seeks to solve these and other problems.

### SUMMARY OF EXAMPLE EMBODIMENTS

In one embodiment, a method of nucleic acid extraction utilizes small magnetizable beads (e.g., iron, cobalt, nickel, etc.) that react to the presence of a magnetic field by participating in a paramagnetic flux. The magnetizable beads clump together in a mass inside of a reaction vessel. The reaction vessel can be different shapes and sizes, but it is typically cylindrical like a test tube. A magnet is positioned proximal to the reaction vessel with the beads inside. By rotating the magnet around the tube (or spinning the tube), the beads will follow the magnet around the tube due to the magnetic flux, creating a grinding or macerating effect while mixing the contents. When biological samples are also placed into the reaction vessel with the magnetizable beads, they become homogenized in the reaction vessel as the magnet moves around the vessel.

### BRIEF DESCRIPTION OF THE DRAWINGS

FIG. 1 illustrates a side elevation view of a reaction vessel with magnetizable beads therein;

FIG. 2 illustrates a side elevation view of a system and method of nucleic acid extraction;

FIG. 3 illustrates a top, side perspective view of a system and method of nucleic acid extraction with heater coils;

FIG. 4 is a front, top perspective view of a system and method of nucleic acid extraction; and

FIG. 5 is a front, top perspective view of a system and method of nucleic acid extraction.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As previously discussed, there is a need for an extraction method that produces highly purified nucleic acids in just a matter of minutes, regardless of the specimen type. The system and method disclosed herein solves these problems and others.

The powerhouse of PCR is real-time PCR (RT-PCR). This is where each cycle of PCR (there are typically 40 cycles per run) is measured for growth or amplification of PCR product. The amplification is an exponential process that produces billions of copies of product, making it one of the most sensitive technologies available for molecular diagnostics. With all of its power and advantages, sample preparation remains a slow process, bottlenecking the overall RT-PCR process. For example, it usually takes 15-60 minutes to process a specimen to extract the purified DNA or RNA. Depending on specimen type, that time could be significantly longer. For example, it is not uncommon to perform a 12-24 hour enzymatic digestion of a chunk of tissue from a patient in order to get the specimen ready to begin the extraction process. Some specimens are very difficult to process because releasing the nucleic acids from them may be difficult. As discussed earlier, Bacillus anthracis spores are one such specimen that is difficult in releasing nucleic acids. The system and method of nucleic acid extraction disclosed herein solves these problems and others.

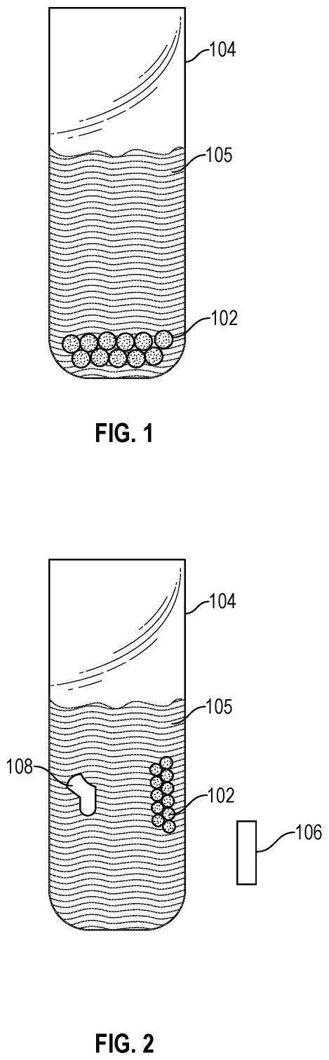

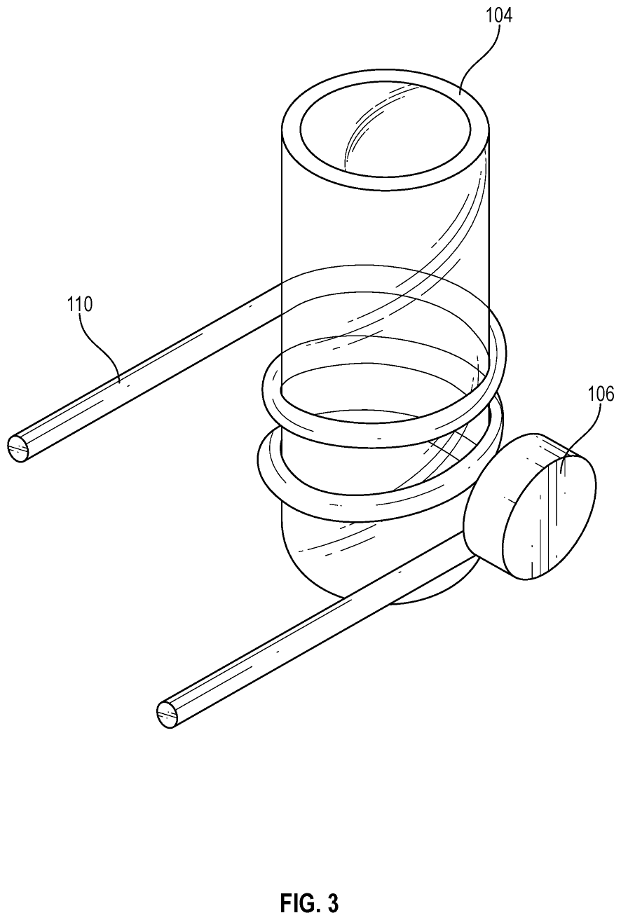

In some embodiments, as shown in FIGS. 1-3, a system and method of nucleic acid extraction comprises placing a plurality of small magnetizable beads 102 into a reaction vessel 104 (e.g., test tube) and rotating at least one magnet 106 around the reaction vessel 104 within sufficient proximity to the reaction vessel 104 that the small magnetizable beads 102 are influenced by the magnetic field (e.g., paramagnetism). By rotating the magnet 106 around the reaction vessel 104, the magnetizable beads 102 will follow the magnet 106 around the tube due to the magnetic flux. This effect causes a grinding or macerating effect, as well as a swirling and mixing of the accompanying solution. For example, when biological samples 108 are placed into the reaction vessel 104 with the magnetizable beads 102, they are ground and swirled in the reaction vessel 104 as the magnet 106 moves around the reaction vessel 104, homogenizing the mixture. By macerating and homogenizing the sample 108 in the reaction vessel 104 using the magnetizable beads 102, the sample 108 is made ready for PCR in a fraction of the time.

Once the sample 108 has been processed by the magnetizable beads 102, the sample 108 can go through the numerous available extraction protocols known in the art. One such example is chaotropic salt guanidine hydrochloride. This allows the sample to be processed and ready for use in just a matter of minutes. For example, laboratory results have yielded highly pure DNA and RNA in just two to three minutes, including the pretreatment step, regardless of sample type. This is a significant improvement over the prior art, which currently requires around 15-60 minutes. Spores may also be processed using these methods.

Depending on the sample 108 being processed, in some embodiments, the magnetizable beads 102 can be coated (with a protectant and/or for added abrasiveness), non-coated, and have rough (e.g., bumpy, spiky, textured, etc.) or smooth surfaces to increase or decrease their abrasiveness, respectively. As a result, regardless of the sample 108 being processed, a user may select the appropriate magnetizable beads 102 so as to maintain a processing time of only a few minutes—a dramatic improvement over the prior art. For example, a user may choose a more abrasive magnetic bead 102 so as to more easily macerate a spore. Additionally, the magnetizable beads 102 may be various sizes (i.e., diameters) for handling large and small samples 108, which may be simultaneous (e.g., tissue samples and blood cells). It will be appreciated that the magnetizable beads 102 may comprise various metals/compositions as long as they react to the magnet 106.

As shown in FIG. 3, a heater element 110 may be coiled around the reaction vessel 104, with the magnet 106 rotating around the heater element 110. By combining a heater element 110 with the magnetizable beads 102 and magnet 106, processing of samples 108 is greatly accelerated. While the heater element 110 is illustrated as coiled around the reaction vessel 104, it will be appreciated that it need not be coiled around the reaction vessel 104 and that other configurations and heater elements may be used as long as the magnetic field between the magnet 106 and the magnetizable beads 102 is not disrupted.

In addition to disrupting a specimen, the system and method disclosed herein performs another critical step in processing a sample 108, which is the homogenization of the solution to remove the chemical gradients that can form in a reaction vessel 104. When an extraction is performed manually, an instrument called a vortex mixer is often used at various steps to ensure that the extraction solutions and included specimen sample are completely mixed (i.e., homogenized). This is necessary to maximize the yield in processing. The system and method disclosed herein consolidates these steps by disrupting a specimen while simultaneously mixing the solution to create a homogenized sample, using the magnetizable beads 102. This is important when the system disclosed herein is embodied in a small/portable automated device where a vortex mixer may not be feasible. In order to aid the mixing of the contents, the sample vessel 104 may comprise interior ridges (e.g., vertical ridges on the interior wall of the sample vessel 104) to disrupt the flow of the magnetizable beads as they rotate around the inner wall. This induces additional movement of the magnetizable beads, ensuring a homogenized solution.

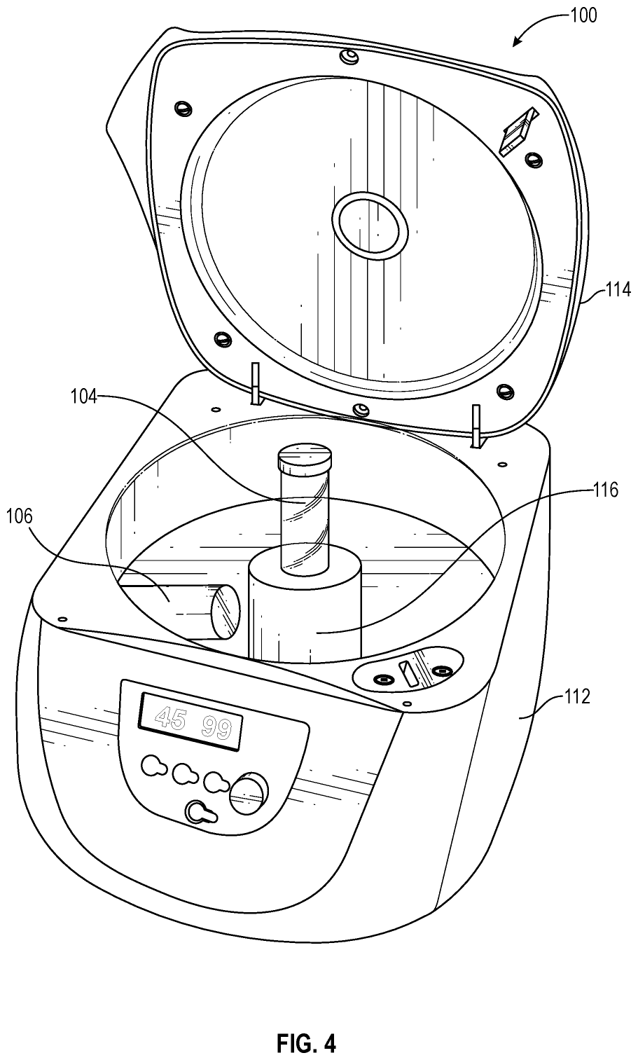

As shown in FIG. 4, the system of nucleic acid extraction 100 disclosed herein may comprise a housing 112 having a lid 114, a cartridge receiver 116 for receiving a sample vessel 104, one or more magnets 106 in sufficient proximity to the cartridge receiver 116 so as to exert a magnetic field on a plurality of magnetizable beads 102 (not visible in this view) within the sample vessel 104, and a motor (located inside the housing 112) for spinning the cartridge receiver 116, thereby spinning the sample vessel 104. In this scenario, a user may close and seal the sample vessel 104, place it into the cartridge receiver 116, close the lid 114, and start the device (e.g., turn on the device, actuating the motor). Due to the magnetizable beads 102 within the sample vessel 104, the sample 108 is macerated and the solution 105 therein is homogenized. It will be appreciated that the solution may comprise those known in the art, including preservatives (e.g., guanidinium, Chelex, Phenol, or other solutions to stabilize biological molecules). In some embodiments, the cartridge receiver 116 may further comprise a heater element, allowing a user to vary the temperature as needed.

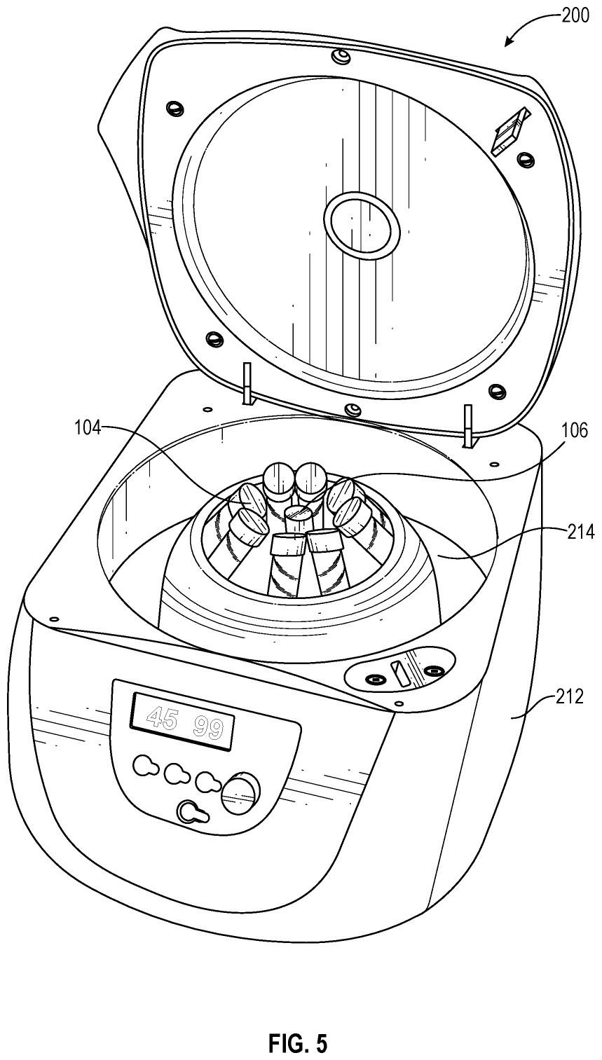

In some embodiments, as shown in FIG. 5, one or more magnets 106 may be fixed in the center of a housing 212, with a cartridge receiver 214 comprising a plurality of slots for receiving cartridges (e.g., sample vessels 104) spinning around the magnet 106, thereby mixing the plurality of sample vessels simultaneously. In order to ensure that the samples are sufficiently macerated and homogenized, each sample vessel 104 may be coupled to the cartridge receiver 214 via a plurality of bearings (not visible in this view). The bearings allow each sample vessel to rotate within the receiving cartridge 214, ensuring that the contents of each are sufficiently homogenized. Although similar in appearance to a centrifuge, rather than the sample vessel remaining 104 remaining stationary within the receiving cartridge 214, resulting in separation of contents (e.g., supernatant and pellet), the system of nucleic acid extraction 200 homogenizes the contents of each sample vessel 104 while also allowing for centrifugation of the sample, if needed.

While described as the cartridge receiver spinning, it will be appreciated that other configurations may also achieve the same result without departing herefrom. For example, in some embodiments, the cartridge receiver is stationary while the magnet spins around the cartridge receiver. In some embodiments, the cartridge receiver and the magnet move in opposing directions. In some embodiments, the permanent magnets are replaced with electromagnets. In some embodiments, there is a stator array or circular disk of electromagnets that can be energized sequentially to pull the beads around the sample tubes 104.

Therefore, as understood from the foregoing, the system and method of nucleic acid extraction disclosed herein dramatically accelerates sample preparation for use with PCR, overcoming problems in the prior art discussed above herein.

It will be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A method of nucleic acid extraction, comprising:
one or more magnetizable beads placed in a sample vessel;
a specimen and a solution added to the sample vessel;
a magnet positioned proximal to the sample vessel so that the magnetic field interacts with the one or more magnetizable beads;
wherein the magnet is configured to revolve around the sample vessel, the one or more magnetizable beads thereby homogenizing the sample and solution in the sample vessel.

2. The method of claim 1, further comprising a heater element positioned proximal to the sample vessel.

3. The method of claim 1, wherein the magnetizable beads comprise a coating.

4. The method of claim 3, wherein the coating is protective.

5. The method of claim 1, wherein the sample vessel comprises ridges.

6. The method of claim 1, wherein the solution comprises a preservative.

7. The method of claim 1, wherein the sample vessel is received within a cartridge receiver.

8. The method of claim 7, wherein the cartridge receiver is within a housing.

9. A method of nucleic acid extraction, comprising:
a plurality of magnetizable beads placed in a sample vessel;
a specimen and a solution added to the sample vessel;
a magnet positioned proximal to the sample vessel so that the magnetic field interacts with the magnetizable beads;
wherein the sample vessel is configured to spin in relation to the magnet, the magnetizable beads thereby homogenizing the sample and solution in the sample vessel.

10. The method of claim 9, further comprising a heater element positioned proximal to the sample vessel.

11. The method of claim 9, wherein the magnetizable beads comprise a coating.

12. The method of claim 11, wherein the coating is protective.

13. The method of claim 9, wherein the sample vessel comprises ridges.

14. The method of claim 9, wherein the solution comprises a preservative.

15. The method of claim 9, wherein the sample vessel is received within a cartridge receiver.

16. The method of claim 7, wherein the cartridge receiver is within a housing.

17. The method of claim 16, comprising a motor for spinning the cartridge receiver.

18. A method of nucleic acid extraction, comprising:
a plurality of magnetizable beads placed in a sample vessel;
a specimen and a solution added to the sample vessel;
a magnet positioned proximal to the sample vessel so that the magnetic field interacts with the magnetizable beads;
wherein the sample vessel is configured to spin in a first direction and the magnet is configured to revolve around the sample vessel in a second, opposite direction, the magnetizable beads thereby homogenizing the sample and solution in the sample vessel.

19. The method of claim 18, wherein the sample vessel comprises ridges.

20. The method of claim 18, wherein the magnetizable beads each comprise a protective coating.
